(19) United States
(12) Patent Application Publication
(10) Pub. No.: US 2026/0198820 A1
(43) Pub. Date: Jul. 16, 2026

(54) SYSTEM FOR AND METHOD OF EYE TRACKING

(71) Applicant: The General Hospital Corporation, Boston, MA (US)

(72) Inventors: Xue-Jun Kong, Charlestown, MA (US); Kenneth K. Kwong, Charlestown, MA (US); Raymond K. Wang, Charlestown, MA (US)

(21) Appl. No.: 19/135,032

(22) PCT Filed: Dec. 4, 2023
(86) PCT No.: PCT/US2023/082298

§ 371 Date: Jun. 2, 2025

Related U.S. Application Data
(60) Provisional application No. 63/385,953, filed on Dec. 2, 2022.

(51) Int. Cl. A61B 5/16; A61B 5/00

(52) U.S. Cl. CPC A61B 5/163; A61B 5/0077; A61B 5/168; A61B 5/7282; A61B 5/742

(57) ABSTRACT

Systems and methods for identifying a change in visual fixation of an individual over time implement and/or include: collecting a data set collected indicative of an individual's visual fixation with respect to a visual stimulus as determined by an eye tracking device; extracting one or more eye tracking metrics from the data set; generating, via software executing on a processor, an indication of at least one of a diagnosis or subtype indication based on the one or more eye tracking metrics.

### CROSS-REFERENCE TO RELATED APPLICATIONS

This application claims the benefit of and priority to U.S. Provisional Patent Application No. 63/385,953, filed on Dec. 2, 2022, entitled “System for and Method of Eye Tracking,” the entire contents of which are herein incorporated by reference for all purposes.

### TECHNICAL FIELD

This disclosure relates to the field of eye tracking for screening for disorders, such as, for example, autism spectral disorder (ASD).

### BACKGROUND

Eye tracking (ET) has been explored as an early and objective screening method for various disorders, including autism spectral disorder (ASD).

Early diagnosis and intervention significantly impact the prognosis of individuals with Autism Spectral Disorder (ASD). Diagnostic and Statistical Manual of Mental Disorders, Fifth Edition (DSM-5) and Autism Diagnostic Observation Schedule, Second Edition (ADOS-2) have been developed to provide an official diagnosis. However, these comparative methods are usually lengthy, difficult, and costly. The shortage of the resources for such evaluation often delays the diagnosis and further treatment. The mean age of diagnosis is still 4-5 years despite advances and efforts. Moreover, racial ethnic minority, low-income, and/or non-English speaking children with ASD are diagnosed later than white children using comparative methods.

There is a need for a metrics system with higher sensitivity and specificity that can diagnose and/or differentiate disorders, including better differentiating individuals with ASD from those without ASD. Early diagnosis and early intervention can significantly improve the prognosis of children with ASD.

### SUMMARY

The present disclosure overcomes these and other drawbacks by providing systems and methods for eye tracking as a screening method, including screening for ASD. The systems and methods described herein provide improved sensitivity and specificity relative to comparative systems and methods.

According to one aspect of the present disclosure, a method for identifying a change in visual fixation of an individual over time is presented. The method comprises collecting a data set collected indicative of an individual's visual fixation with respect to a visual stimulus as determined by an eye tracking device; extracting one or more eye tracking metrics from the data set; and generating, via software executing on a processor, an indication of at least one of a diagnosis or subtype indication based on the one or more eye tracking metrics.

According to another aspect of the present disclosure, a system for identifying a change in visual fixation of an individual over time is presented. The system comprises an eye tracking device configured to track eye movement of the subject while the subject watches a visual stimulus; a processor coupled with the eye tracking device and containing program instructions that, when executed, cause the system to: collect a data set from the eye tracking device, the data set being indicative of an individual's visual fixation with respect to the visual stimulus, extract one or more eye tracking metrics from the data set, and generate an indication of at least one of a diagnosis or subtype of ASD based on the one or more eye tracking metrics.

According to another aspect of the present disclosure, a non-transitory computer-readable medium is provided. The non-transitory computer-readable medium stores instructions that, when executed by a processor of a system, cause the system to perform operations comprising collecting a data set collected indicative of an individual's visual fixation with respect to a visual stimulus as determined by an eye tracking device; extracting one or more eye tracking metrics from the data set; and generating, via software executing on a processor, an indication of at least one of a diagnosis or subtype of ASD based on the one or more eye tracking metrics

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an example in more detail than is necessary for a fundamental understanding of the teachings of the disclosures. In the drawings:

FIG. 1A illustrates an example image display in accordance with various aspects of the present disclosure.

FIG. 1B illustrates a graph of an example of gaze data for the image display of FIG. 1A.

FIG. 2 illustrates a graph of an example of ADOS-2 scores.

FIG. 3 illustrates another graph of an example of gaze data for the image display of FIG. 1A.

FIG. 4A illustrates an example video frame with a first overlay in accordance with various aspects of the present disclosure.

FIG. 4B illustrates an example video frame with a second overlay in accordance with various aspects of the present disclosure.

FIG. 4C illustrates an example video frame with a third overlay in accordance with various aspects of the present disclosure.

FIG. 5 illustrates a graph of an example of gaze data for the image display of FIG. 4A.

FIG. 6 illustrates another graph of an example of gaze data for the image display of FIG. 4A.

FIG. 7 illustrates an example video frame in accordance with various aspects of the present disclosure.

FIG. 8 illustrates an example video frame in accordance with various aspects of the present disclosure.

FIG. 9A illustrates a graph of an example of eye tracking metrics in accordance with various aspects of the present disclosure.

FIG. 9B illustrates a graph of an example of eye tracking metric correlation in accordance with various aspects of the present disclosure.

FIG. 10 illustrates an example of an eye tracking system in accordance with various aspects of the present disclosure.

FIG. 11 illustrates an example of an eye tracking method in accordance with various aspects of the present disclosure.

### DETAILED DESCRIPTION

In the following detailed description, reference is made to the accompanying drawings in which specific examples are shown by way of illustration. These examples are described in sufficient detail to enable those of ordinary skill in the art to practice the disclosure. It should be understood, however, that the detailed description and the specific examples, while indicating examples of embodiments of the disclosure, are given by way of illustration only and not by way of limitation. From this disclosure, various substitutions, modifications, additions rearrangements, or combinations thereof within the scope of the disclosure may be made and will become apparent to those of ordinary skill in the art.

Unless otherwise indicated, the various features illustrated in the drawings may not be drawn to scale. The illustrations presented herein are not necessarily intended to be actual views of any particular method, device, or system, but are merely idealized representations that are employed to describe various embodiments of the disclosure. Accordingly, the dimensions of the various features as illustrated may be arbitrarily expanded or reduced for clarity. In addition, some of the drawings may be simplified for clarity. Thus, the drawings may not depict all of the components of a given apparatus (e.g., device) or method. In addition, like reference numerals may be used to denote like features throughout the specification and figures.

It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. Also, unless stated otherwise a set of elements may comprise one or more elements.

As used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “only one of,” or “exactly one of.” For example, a list of “only one of A, B, or C” indicates options of: A, but not B and C; B, but not A and C; and C, but not A and B. In contrast, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more A, one or more B, and one or more C. Similarly, a list preceded by “a plurality of” (and variations thereon) and including “or” to separate listed elements indicates options of one or more of each of multiple of the listed elements. For example, the phrases “a plurality of A, B, or C” and “two or more of A, B, or C” indicate options of: one or more A and one or more B; one or more B and one or more C; one or more A and one or more C; and one or more A, one or more B, and one or more C.

As will be described herein, the present disclosure provides systems and methods for eye tracking as a screening method for ASD. The eye tracking system described herein includes an imaging device, a controller and display device.

Without wishing to be bound to any particular theory of operation, ASD individuals have different eye movements and gaze patterns correlated with different attention features, such as difficulties in interpreting gaze cues, preferences to fixate on more highly systemized pictures, decreased attention to faces, and a lack of right hemispheric dominance for face processing.

The metrics system described herein includes but is not limited to area of interest (AOI) switches, attention shifts among AOIs, total gaze point or fixation counts through self-assigned AOI shift pathways (e.g., favored or unfavored pathways, and their difference), and/or AOI vacancy incidences. These eye tracking metrics have significantly higher sensitivity and specificity to differentiate those with ASD from those without ASD, which could serve as biomarkers to help early diagnosis and sub-typing of ASD. In return, these will allow early and targeted intervention and therefore result in a better prognosis for ASD patients, many of whom may otherwise need a life-long care due to social and/or intellectual impairments.

As described in further detail below, this eye tracking metrics system has higher sensitivity and specificity to differentiate ASD individuals from those non-ASD. A correlation of these new metrics with ADOS-2 sub-scales indicates the screening and diagnostic values for ASD using eye tracking.

The present disclosure describes several eye tracking metrics including but not limited to AOI switch incidence, attention shifts among AOIs, AOI shift pathways such as favored and unfavored pathways and their difference, and AOI vacancy incidence. A measurement system is established for these metrics to detect the core deficit of ASD with higher sensitivity and specificity than comparative examples, some of which use fixation, saccades, and pupil size to differentiate ASD individuals from those non-ASD.

The present disclosure further describes the correlation of these metrics with ADOS-2 sub-scales to indicate the screening and diagnostic values for ASD using eye tracking. The non-ASD group used for systems and methods in accordance with the present disclosure are those with high risk for ASD or those with ASD traits, instead of neurotypicals (NT) used in comparative methods. The difference between ASD and non-ASD group is smaller or more subtle, which utilizes more sensitive or specific metrics.

The present disclosure provides non-invasive, rapid, objective, easily applied, quantifiable biomarkers which could consistently appear from early childhood to older age. Eye tracking may be used as an early screening tool for ASD with easy operation and fast conclusion. In the present disclosure, a system is established with a battery of metrics featuring AOI switch counts, AOI shift pathways (including favored AOIs (FAS), unfavored AOI (UAS), and their difference), AOI vacancy incidences to direct reflect joint attention, and/or referential and attentive looking. These applications are more sensitive and specific to differentiate ASD from non-ASD in a narrow gapped high-risk cohort. These applications with tailored paradigm scenarios (typically lasting less than 1 minute) may be embodied as an app using a smartphone, tablet, personal digital assistant (PDA), laptop computer, desktop computer, or other device having an eye tracker device (e.g., a front-facing camera) and a connection to the Internet. The present disclosure may include a built-in calculation system that may offer immediate preliminary results with, where appropriate, subsequent professional services for further evaluations. The central managed remote service could be consistently offered in combination with physician consultation to provide more sensitive and specific objective measurements for ASD features. Thus, the present disclosure provides benefits in in ASD early diagnosis, screening, sub-typing as a reliable tool with easy application and quick conclusion to guide early and targeted interventions.

Further data collections in combination with deep learning and artificial intelligence will improve accuracy to adopt different clinical settings, such as application in early infancy to identify those with high risks to potentially prevent social impairments that might otherwise only emerge at older ages. This will substantially reduce the ASD-related economic burden on public health system and remove potential economic and psychological impacts on many involved families.

For purposes of illustrating the systems and methods of the present disclosure, an example experiment was performed. The following discussion of the study is not intended to limit the scope of the claims.

Methods: In the present disclosure, a battery of new eye tracking metrics are presented including AOI switches, total gaze point or fixation counts through self-assigned AOI shift pathways and AOI vacancy counts (AVC) in toddlers and preschoolers with ASD (n=22) and without ASD (n=17) using paradigm video clips. The correlation between these eye tracking metrics and ADOS-2 subscales, and their cut off scores, were also performed via a linear regression analysis.

Participants: Thirty-nine individuals aged 18-84 months old (i.e., toddlers and preschoolers) participated in the study, the participant recruitment included different ethnic groups to encourage a more ethnically heterogenous study cohort. The enrolled study participants included 15 White (42.8%), 11 Asian (31.4%), and 9 (25.7%) subjects of other races. Twenty-three males and twelve females participated in the study. Sex was defined by sex chromosomes composition: “males” are those individuals who have XY chromosomes, and “females” are those individuals who have XX chromosomes. Individuals were recruited through clinical care clinics and online recruitment sites. All participants were included after being identified as high-risk for ASD by clinicians or caregivers. The high-risk status was confirmed through a phone screening prior to enrollment. Inclusion criteria included one or more of the following: (1) at least one sibling with a clinical diagnosis of ASD; (2) a caregiver or clinician indicated concerns about the child's development of social interaction, play, or other behaviors; and/or (3) the individual scored in the positive range on the Modified Checklist for Autism in Toddlers (M-CHAT). Exclusion criteria included major congenital or genetic disorders or diseases, or behavioral problems that would cause substantial additional stress for the family and/or the child during testing. Individuals with a previous diagnosis of ASD were included, but the examiner was not informed of the diagnosis.

Assessment Instruments and Protocols: The study involved human participants and was reviewed and approved by the Institutional Review Board (IRB) of Massachusetts General Hospital. Informed consent to participate in the study was provided by the participant's legal guardian. Prior to the visit a phone screening was done to determine whether the candidate met inclusion criteria for the study. During the phone screening, some demographic information was recorded. During the visit, 2 different tests were performed. The first test was an eye-tracking study. A Tobie X3-120 eye tracker was used for data collection. Screen resolution was set to 1,024×768 pixels with a sampling frequency of 250 Hz and spatial resolution of 0.03 degrees. The subjects were seated in front of a 22-inch widescreen LCD monitor in a dark and soundproof room. The center of their vision was aligned with the center of the monitor, with an eye-to-monitor distance of 65 cm. Before the short video presentation, eye position correction was performed by having the subjects fixate on a dynamic pink rabbit (five-point calibration). Those who failed to follow the dynamic pink rabbit fixation did not continue in the experiment. After five-point calibration, silent video clips were presented for around 2 minutes. Only the data of the participants who cooperated, completed the whole experiment, and passed the quality check were included in the data analysis.

### Stimuli of Eye Tracking:

The present disclosure discusses several example scenarios and parameters; however, it should be noted that the list is not exhaustive and other scenarios and/or parameters may be used without departing from the scope of the present disclosure. The stimuli consisted of several simple video clips and pictures.

The first video, referred to as video 1, depicted a woman's face and a tablet. The video was 25 seconds long. In the video, a woman was shown on the left side of the screen and a tablet was on the right side of screen. The total 25 seconds were divided into four time blocks based on the following sequence of attention focuses: 1) the woman looked at the user while the tablet showed moving objects, 2) the woman turned off the tablet then the woman looked at the user while the tablet was blank, 3) the woman turned on the tablet again similar to focus 1), and 4) the woman turned off the tablet again similar to focus 2). This video was designed to test joint attention, and in NT individuals it was expected that the attention would shift from tablet to woman, back and forth, one after another.

The second video, referred to as video 2, depicted a woman's face. This video was 10 seconds and consisted of a woman mouthing the alphabet (without sounds). The eyes, which play an important role in social communication and emotional expression, constituted one AOI and the mouth, which represents functional responses related to (early) stages of normative language development, constituted another AOI.

The third video, referred to as video 3, consisted of a woman's sad face on the left side of the screen and the same woman with a neutral face on the right side of the screen. The video lasted 10 seconds. After 5 seconds the faces switched position, such that the neutral face was on the left side of the screen and the sad face on the right side of the screen for 5 seconds.

The fourth video, referred to as video 4, depicted a person walking upright on one side of the screen, and the same figure rotated 180 degrees (with the person appearing to walk upside down) was shown on the other side. Each figure constituted an AOI.

ADOS-2: the ADOS module used, which was determined based on the age of the participant, took around 30 minutes to an hour to finish. It contains five modules that are differentiated by participant's developmental and language levels (Module T, 1, 2, 3, and 4). Every ADOS-2 module ends with a diagnostic algorithm(s) that consists of selected items that have been chosen to maximize diagnostic sensitivity and specificity. In this study, the ADOS-2 was administered by professionally trained investigators, in consultation with a certified ADOS trainer, as needed. Following the standardized algorithm of corresponding modules, the composite score, social affect (SA), and restrictive and repetitive behavior (RRB) sub-scores were all recorded for each subject on a score booklet and scored right after the visit. The ADOS-2 Modules 1-3 included a standardized calibrated severity score (CSS) from 1-10. ADOS scores were converted to total CSS, SA CSS and RRB CSS. ADOS-2 was administered by two different professionally trained administrators, and eye tracking was administered by three different professionally trained administrators. The overall evaluation time was around 1 hour.

Statistics: For the ET, the raw data was downloaded from Tobii Pro. Trials with less than 25% screen-looking time (% of trials in the ASD group and % of trials in the non-ASD group) were excluded from the final data analysis. The study also excluded children whose valid trial number was less than 50% (i.e., 6 trials).

The correlation between total gaze count (TGC), fixation duration, fixation count and saccade of AOIs, switch and shift of AOIs, pupil size and ADOS sub-scores were calculated for participants of ASD and non-ASD. ADOS scores are converted to total CSS, SA CSS, and RBB CSS for comparison across Module-T, 1 and 2. The sensitivity and specificity of each eye tracking score in predicting the final diagnosis were computed, and the cut-off scores with the desired sensitivity and specificity were picked for separating the ASD and non-ASD group.

TGC, ASC, FAS, AVC of each subject were compared between ASD and non-ASD groups and were examined using a Wilcoxon rank-sum test. Discriminant Analysis was performed to rank the AOIs by their ability to categorize the subject by their ASD severity level. Data was analyzed using R/R-Studio. For video 1 AOI shift analysis, the total 25s period was divided into 4 attention shift time blocks as described above, and TGC, ASC, FAS, AVC within, or cross different attention time blocks were compared between ASD and non-ASD groups. The correlation between TGC, FAS, AVC, ASC, and ADOS total/sub-scores were calculated for participants of ASD and non-ASD. Total CSS, SA CSS, and RRB CSS for comparison across Module-T, 1 and 2, sensitivity and specificity of each ET metrics score in predicting the final diagnosis were computed, and the cut-off scores with the optimal sensitivity and specificity were picked for separating the ASD and non-ASD group.

### Results:

### 1. Demographic and Clinical Profile

For the study cohort, 39 high risk children for ASD were included in data analysis. The participants included 22 ASD children and 17 non-ASD children. Among them 25 males and 14 females; 15 White (42.8%), 11 Asian (31.4%), and 9 (25.7%) subjects of other races. Table 1 shows the demographic and clinical features of all the participants. There were no significant differences in age or gender between ASD and non-ASD in the two groups; however, their ADOS-2 total and sub-scores were significantly different as expected (Table 1).

TABLE 1
Summary of study participant demographics and ADOS-2 scores.

| | ASD (mean (SD)) | | Non-ASD (mean (SD)) | |
|---|---|---|---|---|
| Age (years) | 3.8 | (1.66) | 3.3 | (1.77) |
| ADOS-2 Total | 7.77 | (1.8) | 4.47 | (2.32) |
| ADOS-2 SA | 8 | (1.72) | 5.41 | (2.9) |
| ADOS-2 RRB | 7.09 | (1.97) | 3.18 | (2.24) |

Sex (count)

| | | |
|---|---|---|
| Male | 16 | 9 |
| Female | 6 | 8 |

Race/Ethnicity (count)

| | | |
|---|---|---|
| White | 8 | 9 |
| African American | 2 | 0 |
| Asian | 7 | 6 |
| Hispanic | 3 | 0 |
| Multi-racial | 2 | 2 |

### 2. The Difference of FAS and AVC Between ASD and Non-ASD Children.

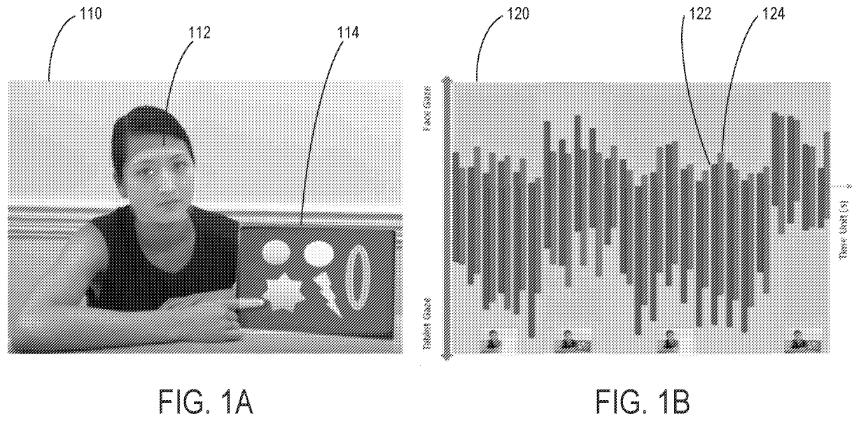

FIG. 1A illustrates a video image 110 that has a first area of interest (AOI) 112 and a second AOI 114. As illustrated in one embodiment, the first AOI 112 is a woman's face and the second AOI 114 is a tablet. Video 1 (25s) contains a woman (AOI-1 112 being her face) on the left side of the screen and a tablet (AOI-2 114) on the right side of the screen (see FIG. 1A). The video elapsed a total of 25s divided into four-time blocks 1-2-3-4 as described above in the protocol (see FIG. 1B): Block 1 is when the tablet was on with pictures moving, meant to draw subjects' attention to watch; Block 2 is when the woman suddenly turned off the tablet then stared at the user, and subjects were expected to turn and look at the woman's face, wondering what is going on at this point; Block 3 is when the woman turned on the tablet again; and Block 4 is when the woman turned off the tablet again. The attention shifts were expected during tablet on-off-on-off. As can be seen in FIG. 1B, the graph 120 shows the gaze data of a first group (non-ASD individuals) 122 and a second group (ASD individuals) 124. The blue bars represent the TGC of non-ASD group, the red bars represent the TGC of ASD group; Green colored areas are the FAS pathway which would be expected of NT subjects following the sequence of tablet-face-tablet-face vs the opposite. Pink colored areas are unfavored attention shift (UAS).

Both the gaze points and fixation time were analyzed and significant differences between groups and time blocks had been observed. Table 2 illustrates the gaze points and fixation time vs time block, AOI, groups, and p values between time blocks, which indicated two groups following the same attention shift pattern while non-ASD goes further in this direction with statistical significance (p<0.05).

TABLE 2
Attention shift correlation vs time blocks.

| Time Shift | | | Gaze | Fix Time |
|---|---|---|---|---|
| Blocks | Group | AOI | p Value | p Value |
| 1 vs 2 | Non-ASD | Screen | <0.0005 | <0.05 |
| | | Face | <0.005 | <0.005 |
| | | Tablet | <0.0005 | <0.025 |
| | ASD | Screen | <0.0005 | <0.025 |
| | | Face | <0.1 | <0.15 |
| | | Tablet | <0.005 | <0.05 |
| 2 vs 3 | Non-ASD | Screen | <0.2 | <0.0005 |
| | | Face | <0.2 | <0.5 |
| | | Tablet | <0.0005 | <0.0005 |
| | ASD | Screen | <0.025 | <0.0005 |
| | | Face | <0.025 | <0.05 |
| | | Tablet | <0.0005 | <0.0005 |
| 3 vs 4 | Non-ASD | Screen | <0.2 | <0.0005 |
| | | Face | <0.2 | <0.2 |
| | | Tablet | <0.0005 | <0.0005 |
| | ASD | Screen | <0.025 | <0.0005 |
| | | Face | <0.025 | <0.5 |
| | | Tablet | <0.0005 | <0.0005 |

Average gazes and fixation time on overall and each AOI time block, favored and unfavored AOIs cross time blocks, AOI vacancy counts cross time block of ASD and non-ASD groups are summarized in Table 3, which shows that the ASD group has significantly reduced gaze count and fixation time in overall favored AOIs cross different time blocks compared with non-ASD group (p<0.0005) and significantly increased in overall unfavored AOI cross different time blocks compared with non-ASD group (p<0.05 for fixation time p<0.025 for gaze count. The difference (favored AOI minus unfavored AOI) is further apart between two groups (p<0.0005). Furthermore, attention on neither AOI could negatively correlate with such social function. These attention occasions are referred to as AOI vacancy counts (AVCs). The ASD group also has very significantly more AOI vacancy counts than the non-ASD group (p<0.001 for fixation time p<0.0005 for gaze count).

TABLE 3
Average gazes with std on each AOI of two groups.

| Time | | Non-ASD | ASD | Gaze p | Non-ASD | ASD | Fix Time |
|---|---|---|---|---|---|---|---|
| Block | AOI | Gaze | Gaze | Value | Fix Time | Fix Time | p Value |
| Overall | Screen | 1868. ± | 1729. ± | <0.1 | 1556. ± | 1672. ± | <0.2 |
| | | 1153. | 1890. | | 1496. | 1998. | |
| Time | Screen | 489. ± | 453. ± | <0.025 | 371. ± | 428. ± | <0.2 |
| Block 1 | | 229. | 283. | | 390. | 525. | |
| | Face | 38.0 ± | 73.0 ± | <0.05 | 18.5 ± | 51.7 ± | <0.025 |
| | | 88.9 | 163. | | 60.7 | 132. | |
| | Tablet | 259. ± | 203. ± | <0.05 | 151. ± | 132. ± | <0.25 |
| | | 241. | 252. | | 215. | 199. | |
| Time | Screen | 319. ± | 266. ± | <0.0005 | 273. ± | 293. ± | <0.5 |
| Block 2 | | 175. | 212. | | 229. | 322. | |
| | Face | 90.0 ± | 42.8 ± | <0.01 | 54.6 ± | 32.2 ± | <0.1 |
| | | 119. | 101. | | 92.1 | 90.8 | |
| | Tablet | 99.7 ± | 103. ± | <0.5 | 82.1 ± | 83.6 ± | <0.5 |
| | | 123. | 161. | | 112. | 145. | |
| Time | Screen | 807. ± | 708. ± | <0.005 | 671. ± | 692. ± | <0.5 |
| Block 3 | | 530. | 560. | | 631. | 794. | |
| | Face | 66.0 ± | 99.1 ± | <0.1 | 50.5 ± | 80.5 ± | <0.1 |
| | | 178. | 255. | | 170. | 225. | |
| | Tablet | 491. ± | 299. ± | <0.0005 | 356. ± | 213. ± | <0.0005 |
| | | 507. | 444. | | 444. | 373. | |
| Time | Screen | 254. ± | 303. ± | <0.0005 | 241. ± | 258. ± | <0.5 |
| Block 4 | | 207. | 601. | | 249. | 352. | |
| | Face | 90.6 ± | 78.6 ± | <0.5 | 73.5 ± | 66.2 ± | <0.5 |
| | | 136. | 139. | | 133. | 128. | |
| | Tablet | 39.4 ± | 35.5 ± | <0.5 | 27.3 ± | 21.9 ± | <0.5 |
| | | 87.3 | 81.7 | | 64.2 | 68.0 | |
| AOIs | Face | 284. ± | 293. ± | <0.5 | 197. ± | 231. ± | <0.2 |
| | | 586. | 671. | | 513. | 589. | |
| | Tablet | 889. ± | 641. ± | <0.0005 | 616. ± | 450. ± | <0.005 |
| | | 1035. | 975 | | 889. | 811. | |
| Favored | UFF | 243. ± | 310. ± | <0.025 | 178. ± | 238. ± | <0.05 |
| AOIs | | 523. | 694. | | 454. | 602. | |
| | FV | 931. ± | 624. ± | <0.0005 | 635.0 ± | 443. ± | <0.0005 |
| | | 1043. | 964. | | 912. | 803. | |
| Difference | FV − UF | 688. ± | 313. ± | <0.0005 | 457. ± | 205. ± | <0.0005 |
| | | 1223. | 1269 | | 1028 | 1058 | |
| AOI | | 7.18 ± | 10.4 ± | <0.0005 | 11.1 ± | 13.8 ± | <0.001 |
| Vacancy | | 2.41 | 2.58 | | 3.18 | 2.13 | |

The TGC was analyzed for both ASD and non-ASD groups in two AOIs across the different time blocks (see Table 4A). This shows that non-ASD children showed significant TGC differences across time blocks 1→2, 2→3 and 3→4 for both AOIs. Instead, ASD children had no TGC difference during the 1→2 and 2→3 shifts, and only started to show a difference during 3→4 shift for both AOIs; meanwhile, the difference between both subject groups showed significance for 1→2 and 2→3 but not 3→4 (see Table 4A). When FAS pathway and AVC (which is subject's gaze counts on neither AOI) were further investigated, it was found that the ASD group had significantly reduced TGC along FAS (p<0.00001) and significantly increased in AVC (p<0.00001) across different time blocks relative to the non-ASD group (see Table 4B).

TABLE 4A
The comparison of significance of total gaze counts
cross time blocks in ASD vs non-ASD groups

| | | non-ASD | ASD | |
|---|---|---|---|---|
| Time | | p between | p between | p between |
| Block | AOI | time blocks | time blocks | groups |
| 1 → 2 | Face | 0.000011 | 0.8261 | 0.00554 |
| | Tablet | 0.000055 | 0.1004 | 0.1508 |
| 2 → 3 | Face | <0.00001 | 0.1491 | 0.01682 |
| | Tablet | 0.000033 | 0.9966 | 0.01735 |
| 3 → 4 | Face | <0.00001 | 0.000733 | 0.5203 |
| | Tablet | <0.00001 | 0.000039 | 0.1704 |

TABLE 4B
The comparison of favored shifts and vacant
attentions in ASD vs non-ASD groups

| | | Non-ASD TGC | ASD TGC | p |
|---|---|---|---|---|
| AOI | Face | 314. ± 608. | 288. ± 651. | 0.5249 |
| | Tablet | 995. ± 1030. | 789. ± 1043. | 0.002142 |
| FAS | | 1022. ± 1039. | 737. ± 1022 | <0.00001 |
| FAS-UAS | | 735. ± 1281. | 396. ± 1341. | 0.00007 |
| AVC | | 4.53 ± 9.64 | 9.18 ± 12.1 | <0.00001 |

Referring back to FIGS. 1A and 1B, an example of one of the video images 110 from a video is shown and gaze data is shown in graph 120. As video 1 progresses, the participants shift their gaze between the first AOI 112 and the second AOI 114. The gaze data from the 4-time blocks from the two participant groups is shown in graph 120.

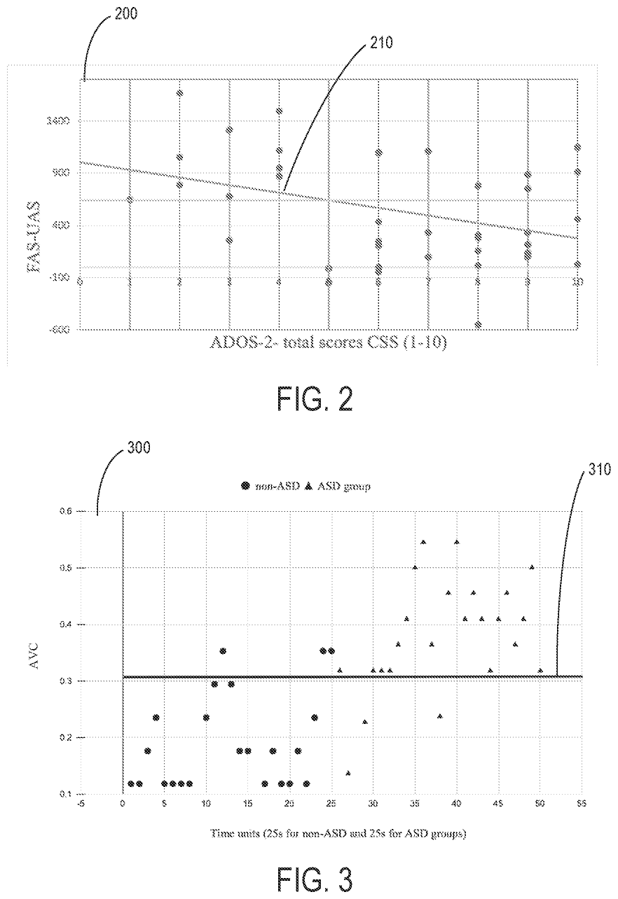

As can be seen in FIG. 2, graph 200 shows a fit line 210. This graph 200 shows example data from a correlation study with regression analysis between the significant eye tracking (ET) index and ASD severity based on Autism Diagnostic Observation Schedule, Second Edition (ADOS-2) scores. Favored AOI shifts-unfavored AOI shifts (FAS-UAS) for video 1 using video image 110 negatively correlated with ADOS-2 total scores (r=−0.373, p=0.01948), SA scores (r=−0.33, p=0.0412) and RRB scores (r=−0.25, p=0.124). When ADOS-2 total CSS cut off score 5 and FAS-UAS cut off score 641.1 were used, the sensitivity was 91%, and the specificity was 72%. The fit line 210 shows a correlation between the two quantities, which represents that different scores correspond to different probabilities of ASD.

Referring to FIG. 3, graph 300 shows the time for non-ASD group shown as circles and ASD groups shown as triangles versus the AVC (AOI vacancy counts) for video 1 as shown in the video image 110 in FIG. 1A. When comparing ASD versus non-ASD groups, AVC had the best sensitivity and specificity among all the ET metrics. As shown in graph 300, the cut off score of 0.305 of AVC vs time unit counts for video 1 achieved sensitivity 88% and specificity 88% to separate ASD from non-ASD. This meant that the subject would not be looking at either AOI as presented, assumed to be not interested, not engaged, or just simply ignoring its existence, which is significantly more in subjects with ASD.

### 3. The Difference of ASC and AVC Between ASD and Non-ASD Children.

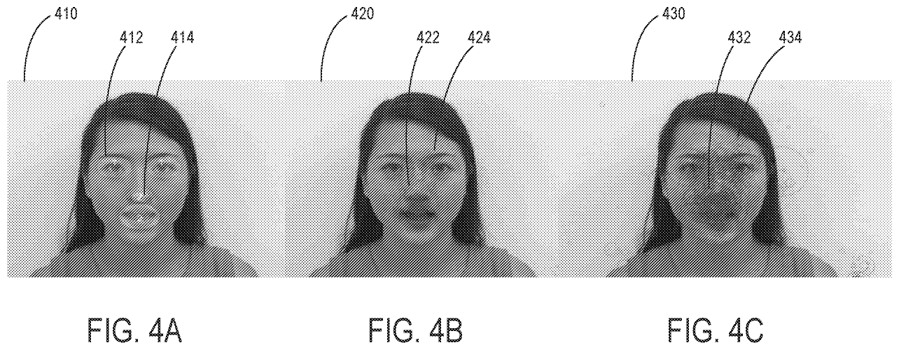

Video 2 (10 seconds) consisted of a woman speaking without sound (mouthing the alphabet). As can be seen in FIGS. 4A-4C, these video frames 410, 420 and 430 are from video 2. This video was used to show the difference of ASC and AVC between ASD and non-ASD children. Two AOIs were defined: AOI-1 412 was defined as the eye area and AOI-2 414 was defined as the mouth area (see FIG. 4A);

In FIGS. 4B and 4C, the subjects' TGC and ASC (i.e., are the switches between these two AOIs) were analyzed in these AOIs. Red dots represent the ASD group and blue dots represent non-ASD group. FIG. 4B shows TGC and FIG. 4C showed TFT (total fixation time) for both groups. The different density distribution pattern between the ASD (red) and the non-ASD groups (blue) can be seen. The ASD group has more diverse and scattered distribution.

Results for the detailed gaze, fixation and saccade time, pupil size and number of switches between two AOIs (AOI switches), and AOI vacancy counts are summarized in Table 5 below. The ASD group has significantly fewer AOI switches between AOI 1 and 2 (P=0.025), and AOI vacancy counts were found to be significantly higher in the ASD group (p=0.0005) vs non-ASD group, while gaze time, fixation length, saccade time and pupil size were found to be smaller in the ASD group than the non-ASD group. However, the difference was not statistically significant (P>0.05) or marginally significant (p=0.05).

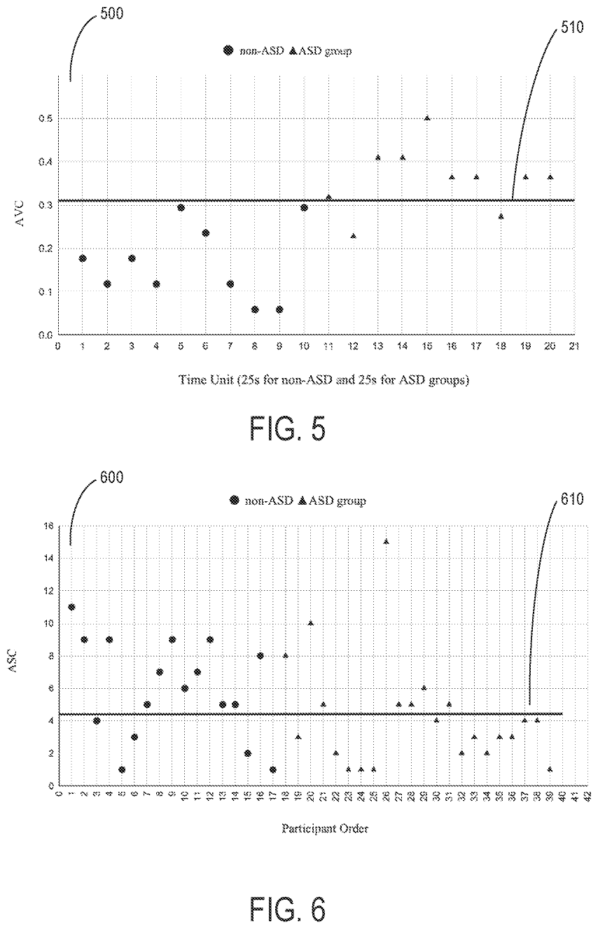

As can be seen in FIG. 5, graph 500 shows the time for non-ASD group shown as circles and ASD groups shown as triangles versus the AVC (AOI vacancy counts) for video 2 as shown in the video frames 410,420 and 430 in FIGS. 4A-4C. When comparing ASD versus non-ASD groups, AVC had the best sensitivity and specificity among all the ET metrics. As shown in graph 500, the cut off score of 0.306 of AVC vs time unit counts achieved sensitivity 100% and specificity 80%. The trend line 510 shows that there is a correlation between which group the participant was in (ASD vs. non-ASD) and the AVC score. This meant that the subject would not be looking at either AOI as presented, assumed to be not interested, not engaged, or just simply ignoring its existence, which is significantly more in subjects with ASD. Reduced TFT (which is correlated with TGC) at people and faces, as well as problems with disengagement of attention, appear to be among the earliest signs of ASD (38).

Referring to FIG. 6, graph 600 shows the participant order for non-ASD group shown as circles and ASD groups shown as triangles versus AOI switch counts (ASC) and a trend line 610. Another important new concept we introduced in this study is the ASC from one AOI to another. The data showed significantly lower ASC in the ASD group than the non-ASD group, in switches from the eye to mouth area of a silently talking woman's face (p=0.045), meaning that subjects with ASDs have less motivation to compare the differences between the AOIs, while the commonly used metrics of TFT and pupil size were found not significantly different under the same stimulus. We found the cutoff 4.5 achieved specificity 71% and sensitivity 64% (p=0.0452). These novel findings indicate that ASC is also more sensitive and specific of an ET feature than TFT and pupil size to differentiate ASD from non-ASD, although not as good as AVC or FAS-UAS. Thinking about the scenario of the silently talking woman's face, normally people will be quickly viewing the mouth area and eyes back and forth (switches) to figure out what she is talking about instead of constantly focusing on eyes or mouth area, this could be referred to as “mind reading”. ASD individuals are less capable of or less interested in mind reading or theory of mind (ToM); therefore, the switches from eyes to mouth are significantly less than those of non-ASD. TOM is the human ability to perceive, interpret, and attribute the mental states of other people, and the alteration of this cognitive function is a core symptom of ASD.

TABLE 5
Fixation, saccade, pupil size and AOI switches

| | | non-ASD | ASD | P value |
|---|---|---|---|---|
| Eye AOI-1 | Gaze count | 113 | 76.3 | 0.15 |
| | Fixation Time | 76.2 | 52.6 | 0.2 |
| | Fixation numbers | 2 | 1.36 | 0.1 |
| | Saccade time | 17.8 | 9.59 | 0.05 |
| | F/S ratio | 0.672 | 0.633 | 0.25 |
| | Pupil Size | 0.286 | 0.258 | 0.05 |
| Mouth AOI-2 | Gaze count | 235.4 | 185.5 | 0.25 |
| | Fixation Time | 199.2 | 157 | 0.25 |
| | Fixation numbers | 2.88 | 1.86 | 0.05 |
| | Saccade time | 24 | 17.6 | 0.15 |
| | F/S ratio | 0.833 | 0.672 | 0.05 |
| | Pupil Size | 0.277 | 0.264 | 0.2 |
| AOI Switch | Between | 103 | 91 | 0.025 |
| | shadow 1 and 2 | | | |
| AOI Vacancy | Gaze Counts | 8.4 + 2.1 | 11 + 1.19 | 0.0005 |

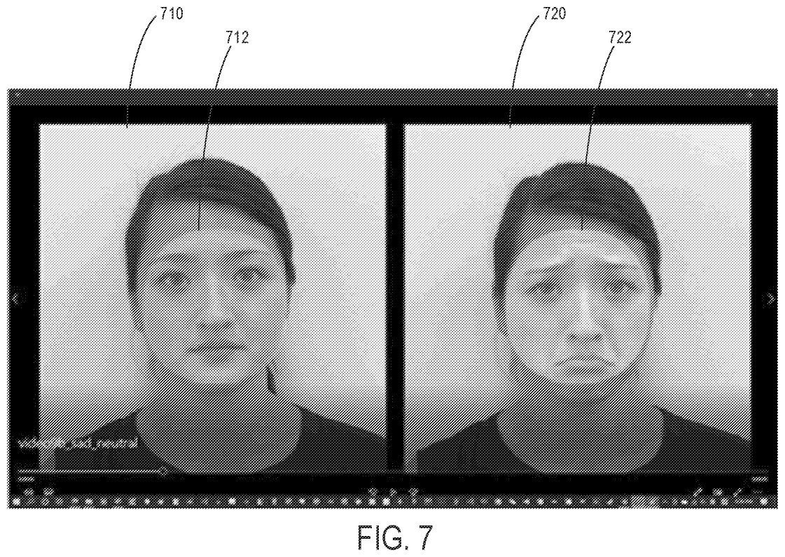

Video 3 (5s): this video consisted of a woman's neutral face on the left side of screen and her sad face on the right side of screen for 5s. In FIG. 7, video frame 710 shows a first expression 712 and video frame 720 shows a second expression 722. The first expression 712 is a neutral face and the second expression is a sad face. The findings of gaze density, fixation, and saccades are summarized in Table 6. The ASD group has less fixation on both the sad face and the neutral face compared with the non-ASD group. The reduced fixation on the neutral face was found to be significant (p=0.025), and the ASD group has significantly reduced numbers of AOI switches between sad and neutral faces (p=0.025) and the ASD group has smaller pupil size which is marginally significant (p=0.05).

TABLE 6
Fixation time and numbers, saccade time and
switches between interested areas of video 3

| | | | non- | | P |
|---|---|---|---|---|---|
| | | | ASD | ASD | value |
| Paradigm 3 | Neutral (Left) | Gaze count | 140 | 81.7 | 0.025 |
| | | Fixation | 107 | 56 | 0.025 |
| | | Time | | | |
| | | Fixation | 1.53 | 0.86 | 0.025 |
| | | numbers | | | |
| | | Saccade time | 21.1 | 14.3 | 0.1 |
| | | F/S ratio | 0.695 | 0.617 | 0.25 |
| | | Pupil Size | 0.265 | 0.241 | 0.05 |
| | Sad (Right) | Gaze count | 155 | 118 | 0.15 |
| | | Fixation | 110 | 79.9 | 0.2 |
| | | Time | | | |
| | | Fixation | 1.29 | 1.14 | 0.25 |
| | | numbers | | | |
| | | Saccade time | 25.4 | 20 | 0.2 |
| | | F/S ratio | 0.622 | 0.641 | 0.25 |
| | | Pupil Size | 0.263 | 0.257 | 0.25 |
| | AOI Switch | Between left | 3.82 | 2.27 | 0.025 |
| | | and right | | | |

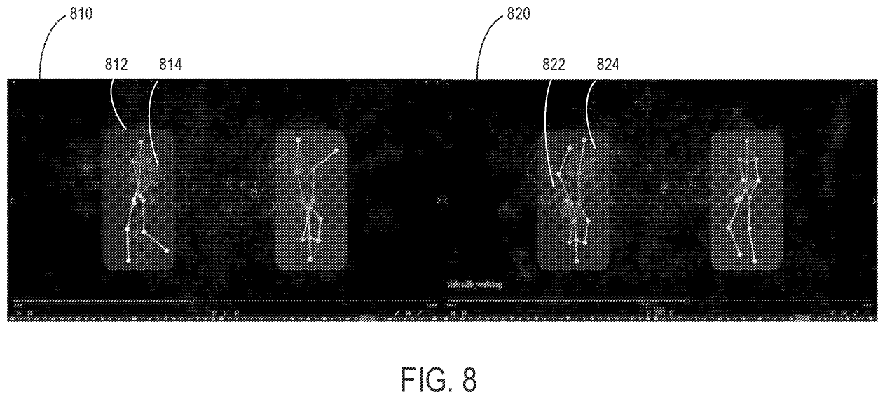

Video 4 (a and b, 5 s each): A point-light display figure of a person walking upright was shown on one side of the screen. On the other side, the same figure was shown rotated 180 degrees, with the person appearing to walk upside down. Each figure was determined as an AOI. FIG. 8 represents paradigm 2a and 2b alternatively. In FIG. 8, video frame 810 shows a gaze density for the first group 812 and a gaze density for the second group 814. Video frame 820 shows a gaze density for the first group 822 and a gaze density for the second group 824. Again, red dots represent ASD group and blue dots represent non-ASD group for gaze density. Video 4 shows a walking skeleton for 10 seconds and then 5 seconds for each scenario. On one side of the screen, a point-light display figure of a person walking upright was shown. On the other side, the same figure was shown rotated 180 degrees, with the person appearing to walk upside down. Each figure was determined as an AOI. Preferential attention to biological motion is a fundamental mechanism facilitating adaptive interaction with other living beings.

The gaze density, fixation, saccade, pupil size and AOI switches between the two figure skeletons are summarized in Table 7 below. It was again found that the ASD group has significant less AOI switches between two walking characters as two separated interested areas (P=0.025) compared with the non-ASD group, and pupil sizes are significantly smaller in ASD group (p=0.025), while gaze density in AOIs and fixation length were found to be not statistically significant (P>0.05). Their fixation/saccade ratio was not statistically different. When video 4b was presented, AOI switches are no longer showing significant difference between two groups, but relative AOI switches are significant (p=0.025), similarly for the pupil size in AOI; meanwhile, the gaze density in AOI of left sided walking upright character was found to be significantly lower in ASD group than non-ASD group (p=0.025) while fixation length was marginally significantly (p=0.05).

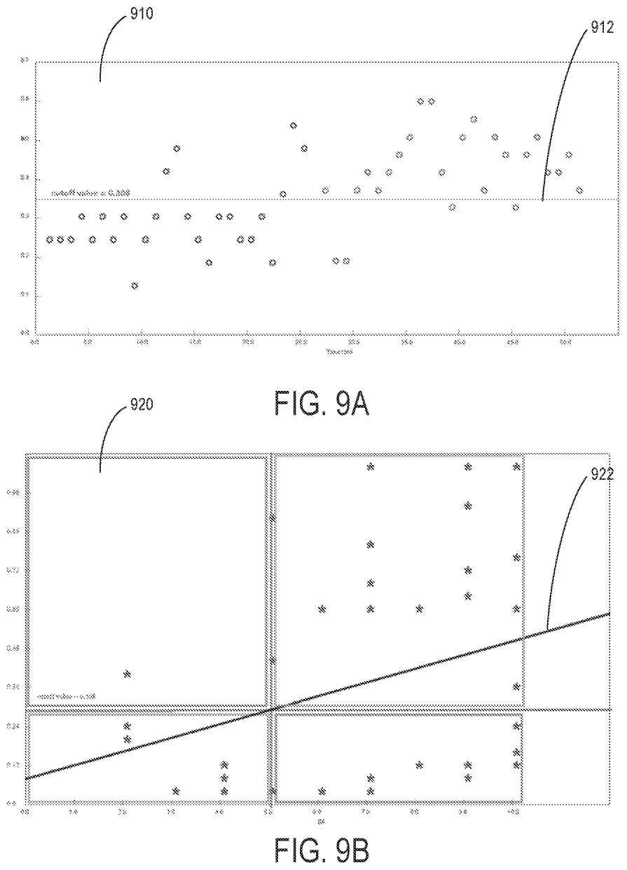

As shown in FIG. 9A, graph 910 is an example plot showing time on the x-axis. The first 25-time units belong to a non-ASD group in blue, and the second 25-time units belong to ASD group in red. Each dot represents an average AOI vacancy incidence of the subjects (y axis) within that time unit. The trend line 912 shows a cuff off value of 0.306.

Referring to FIG. 9B, graph 920 is an example plot of AOI vacancy counts vs SA score, using 0.5 as the SA score cutoff, provided the following result: sensitivity=85.7%, specificity=60.8%, the x-axis represents SA score, y axis represents average AOI vacancy counts cross all time units in one subject with cut off value −0.308, r=0.32, r(tt)=0.3.

TABLE 7
Fixation and saccade switches between interested areas of video 4

| | | Fixation or | non-ASD | ASD | |
|---|---|---|---|---|---|
| | AOI | Saccade | n= | n= | P value |
| Video 4a | Walking | Gaze count | 158 | 165 | 0.25 |
| | upside | Fixation | 118 | 113 | 0.25 |
| | down | length | | | |
| | left | Fixation | 1.24 | 1.27 | 0.25 |
| | side | numbers | | | |
| | | Saccades | 17.8 | 23.5 | 0.2 |
| | | F/S ratio | 0.683 | 0.779 | 0.15 |
| | | Pupil Size | 0.256 | 0.226 | 0.025 |
| | Walking | Gaze count | 232 | 171 | 0.05 |
| | upright | Fixation | 188 | 125 | 0.05 |
| | Right | Length | | | |
| | side | Fixation | 1.41 | 1.18 | 0.2 |
| | | numbers | | | |
| | | Saccades | 24.5 | 23.5 | 0.25 |
| | | F/S ratio | 0.73 | 0.714 | 0.25 |
| | | Pupil Size | 0.253 | 0.225 | 0.025 |
| | AOI | Between left | 4.35 | 2.68 | 0.025 |
| | Switch | and right | | | |
| Video 4b | Walking | Gaze count | 253 | 173 | 0.025 |
| | upright | Fixation | 195 | 131 | 0.05 |
| | Left | length | | | |
| | side | Fixation | 0.537 | 0.396 | 0.05 |
| | | numbers | | | |
| | | Saccades | 23.8 | 18.5 | 0.2 |
| | | F/S ratio | 0.771 | 0.657 | 0.15 |
| | | Pupil Size | 0.247 | 0.222 | 0.05 |
| | Walking | Gaze count | 153 | 132 | 0.25 |
| | upside | Fixation | 126 | 90.8 | 0.1 |
| | down | length | | | |
| | Right | Fixation | 0.348 | 0.275 | 0.15 |
| | side | numbers | | | |
| | | Fixation | 1.18 | 1.05 | 0.25 |
| | | events | | | |
| | | Saccades | 17.7 | 18.3 | 0.25 |
| | | F/S ratio | 0.836 | 0.714 | 0.1 |
| | | Pupil Size | 0.243 | 0.223 | 0.1 |
| | AOI | Between left | 3.35 | 2.82 | 0.15 |
| | Switch | and right | | | |
| | AOI | | 0.0092 | 0.006 | 0.025 |
| | relative | | | | |
| | switch | | | | |

A correlation study was conducted with regression analysis between significant eye tracking index and ASD severity based on ADOS-2 scores. AOI switches were weakly and negatively correlated with RRB score (r=−0.269) while gaze density and fixation are found to be negatively correlated with SA and not RBB in eye area and mouth area. Favored AOI across time block and absent AOI on video were found to also be negatively correlated with SA (r=−0.322 and r=−0.347, respectively) and not correlated with RRB (r<0.1). The cutoff score of each eye tracking metric is presented in Table 8 below.

TABLE 8
Correlation of significant eye tracking metrics and ADOS scores

| | | Fixation | P | SA | | | | |
|---|---|---|---|---|---|---|---|---|
| Paradigm | AOI | or Saccade | value | (R) | RRB(R) | Cutoff | Sensitivity | Specificity |
| Video 1 | Favored | Gaze | 0.0005 | −0.334 | −0.031 | 1826 | 56% | 39% |
| | AOIs | count | | | | | | |
| | Difference | Gaze | 0.0005 | −0.279 | 0.09 | 319 | 68% | 48% |
| | | count | | | | | | |
| | AOI | Gaze | 0.0005 | 0.322 | 0.001 | 7.7 | 80% | 92% |
| | Vacancy | count | | | | | | |
| | Favored | Fixation | 0.0005 | −0.29 | −0.033 | 1551 | 45% | 58% |
| | AOIs | time | | | | | | |
| | Difference | Fixation | 0.0005 | −0.287 | −0.09 | 200 | 72% | 40% |
| | | time | | | | | | |
| | AOI | Fixation | 0.001 | 0.347 | 0.027 | 12.4 | 64% | 88% |
| | Vacancy | time | | | | | | |
| Video 2 | AOI | Gaze | 0.0005 | 0.323 | 0.105 | 9.5 | 80% | 100% |
| | Vacancy | counts | | | | | | |
| | Eye & | AOI | 0.025 | −0.053 | −0.269 | 5.01 | 82% | 59% |
| | mouth | switch | | | | | | |
| Video 3 | Neutral | AOI | 0.025 | 0.1703 | −0.373 | 2.27 | 59% | 76% |
| | & sad | switch | | | | | | |
| | L-neutral | Gaze | 0.025 | −0.42 | −0.256 | 81.7 | 54% | 71% |
| | | count | | | | | | |
| | | Fixation | 0.025 | −0.42 | −0.256 | 80.1 | 64% | 65% |
| | | length | | | | | | |
| | | Fixation | 0.025 | −0.325 | −0.291 | 1 | 68% | 65% |
| | | number | | | | | | |
| Video | FIG. | AOI | 0.025 | −0.024 | −0.304 | 2.68 | 45% | 71% |
| 4a | L/R | Switch | | | | | | |
| | | AOI/gaze | 0.025 | 0.072 | −0.407 | 0.0068 | 73% | 53% |
| | | Pupil | 0.025 | −0.107 | 0.063 | 0.234 | 79% | 76% |
| | | size | | | | | | |
| Video | FIG. L | Gaze | 0.025 | −0.361 | −0.07 | 230 | 73% | 71% |
| 4b | | count | | | | | | |
| | | Fixation | 0.05 | −0.238 | 0.018 | 176 | 77% | 71% |
| | | time | | | | | | |
| | | Saccade | 0.05 | −0.542 | 0.07 | 0.409 | 73% | 59% |
| | | time | | | | | | |
| | FIG. R | Pupil | 0.025 | 0.264 | 0.307 | 0.992 | 84% | 56% |
| | | size in | | | | | | |
| | | AOI | | | | | | |

When the detailed TGC, ASC, and AVC were investigated, it was found that the ASD group had significantly less ASC between AOI 1 and 2 (P=0.0452), and significantly more AVC (p=0.000017) vs the non-ASD group, while TGC was found to be significantly smaller in the ASD group than the non-ASD group for AOI-1 area (P=0.00379), not AOI-2 area (p=0.6537). EP metrics TFT and pupil size (used in comparative systems) were also compared, they all showed no significant difference between two groups (p>0.05), demonstrating that AVC and ASC had significantly higher sensitivity than the comparative metrics (see Table 9).

TABLE 9
Comparison of comparative eye tracking metrics and
eye tracking metrics in accordance with the present
disclosure in ASD vs non-ASD group for video 2

| | | non- | | P- |
|---|---|---|---|---|
| | | ASD | ASD | value |
| Eye | Total gaze | 133.3 | 76.0 | 0.00379 |
| (AOI-1) | count (TGC) | | | |
| | Total fixation | 91.24 | 56.96 | 0.0661 |
| | time (TFT) | | | |
| | Pupil Size | 3.316 | 3.456 | 0.7485 |
| Mouth | Total gaze | 194.5 | 180.4 | 0.6537 |
| (AOI-2) | count (TGC) | | | |
| | Total fixation | 163.1 | 159.2 | 0.902 |
| | time (TFT) | | | |
| | Pupil Size (mm) | 3.475 | 3.142 | 0.474 |
| ASC | Between AOIs | 5.94 | 4.23 | 0.0452 |
| | land 2 | | | |
| AVC | Total gaze counts | 1.812 | 3.950 | 0.000017 |

### 4. Correlation of Significant ET Metrics and ADOS-2 Scores/ASD Diagnosis

A correlation study was conducted with regression analysis between the significant ET index and ASD severity based on ADOS-2 scores. It was found that FAS-UAS for video 1 negatively correlated with ADOS-2 total scores (r=−0.373, p=0.01948), SA scores (r=−0.33, p=0.0412) and RRB scores (r=−0.25, p=0.124). When ADOS-2 total CSS cut off score 5 and FAS-UAS cut off score 641.1 were used, a sensitivity 91% and specificity 72% were obtained.

When ASD (red dots) and non-ASD (blue dots) groups were compared, it was found that AVC had the best sensitivity and specificity among all the ET metrics (including comparative metrics), and the results were consistent in both video 1 (sensitivity 88%, specificity 88%, p<0.00001, cut off score 0.305) and video 2 (sensitivity 100%, specificity 80%, p<0.000045, cut off score 0.306). ASC for video 2 had sensitivity 71%, specificity 64%, p-0.04523, and cut off score 4.5.

The favored AOI shifts in the sequences of different attention focus were found to be significantly less in the ASD group (p<0.05); the switch counts between one AOI and another were significantly less in the ASD group (p<0.05). The AOI vacancy counts were found to be significantly more in the ASD group (p=0.0005) compared with those with non-ASD. Furthermore, the favored AOI shift was found to be negatively correlated with ADOS-2 SA subscale (r=−0.334), and AOI vacancy counts was found to be positively correlated with SA (r=0.347), while AOI switches were found to be negatively correlated with RRB subscale (r=−0.373); the cutoff score of non-AOI counts yield sensitivity 80% and specificity 92%.

### DISCUSSION

In the study described above, a battery of eye tracking metrics were developed and investigated. First, the concept of “favored AOI shift” vs “unfavored AOI shift” was introduced, depending on what the attention shifts normally would be expected or the opposite. This concept was to emphasize the ability of the viewer to adjust his/her attention on the currently appropriate object and to reduce one's attention on the less important object even though it was the “right” one. This is a comprehensive ability. Second, the concept of ASC between two competing targets was introduced. Third, the concept of the AVC as ET metrics to target ASD featuring core deficit to differentiate ASD from those with non-ASD was discussed. These were found to be more sensitive and reliable biomarkers in comparison with previously used TFT and pupil size. Their cut off scores were explored, and a correlation found with ADOS-2 total and SA and RRB sub scores. Each one of these biomarkers and their diagnostic values are discussed below in more detail. The subject population of this study involved all high-risk toddlers or preschoolers for ASD, and we define them ASD vs non-ASD based on the DSM-5. The non-ASD group in this study are not NT peers as used in comparative studies. The difference between ASD and non-ASD groups in this study could be subtle and small which requires more sensitive or specific metrics.

Using video 1 for testing joint attention (JA), the ASD group has significantly less FAS across the time block compared with the non-ASD group. In FAS, the non-ASD group significantly exceeds the ASD group, while in UAS, the ASD group exceeds the non-ASD group with modest significancy. The difference between the favored and unfavored AOIs would result in further difference of the two groups. Across the time blocks, the ASD group showed much less and delayed attention shifts relative to the non-ASD group. These findings consistently reflect poorer or absent JA in ASD. JA is an important human social communication and social cognition skill which is significantly impaired in ASD as a feature presentation and earlier marker. JA started to develop at 5 months, and research found the rates of initiation of JA lower in infants later diagnosed with ASD than in the comparison groups at 10 months of age. In this study, when their correlation with ADOS-2 total and sub-scores was further explored, a negative correlation with TGC of FAS-UAS was found, which was significant for total scores and SA score (p<0.05) but not significant with RRB scores (p>0.05). This indicated a positive correlation between the TGC within favored AOI and social function: the higher SA, the lower social function. A higher correlation is expected if with a neurotypical control instead of high-risk non-ASD control as used in this study. The ET metrics using FAS or FAS-UAS as reliable test for JA feature are promising for ASD early diagnosis.

Another biomarker of interest is the AVC, which was not realized in comparative examples. This represents the TGC that fell outside of all defined AOIs. A significant increase in time unit counts of AVC was found in the ASD group compared with the non-ASD group (p<0.00001 for video 1 and p=0.000017 for video 2), which meant that subjects with ASD tends to be much less attentive than expected and may simply ignore the common interest or “space” out. Furthermore, the cut off score of 0.305 of AVC vs time unit counts for video 1 achieved sensitivity 88% and specificity 88% to separate ASD from non-ASD; similarly for video 2, the cut off score of 0.306 of AVC vs time unit counts achieved sensitivity 100% and specificity 80%. This meant that the subject would not be looking at either AOI as presented, assumed to be not interested, not engaged, or just simply ignoring its existence, which is significantly more prevalent in subjects with ASD. Reduced TFT (which is correlated with TGC) at people and faces, as well as problems with disengagement of attention, appear to be among the earliest signs of ASD. The early reductions in social attention may be infant endophenotypes of social motivation traits related to ASD, while changes in general attention are commonly seen in Attention Deficit and Hyperactive Disorder (ADHD). The gaze-triggered attention was found to be reduced in ASD and influenced by self-relevant gaze cue and associated with symptom severity in ASD. With the excellent cut off score, high sensitivity and specificity, the findings further the potential of ET in early diagnosis as a valuable biomarker.

Another concept introduced in this study is the ASC from one AOI to another. Significantly lower ASC was found in the ASD group than the non-ASD group, in switches from the eye to mouth area of a silently talking woman's face (p=0.045), meaning that subjects with ASDs have less motivation to compare the differences between the AOIs, while the metrics of TFT and pupil size used in comparative methods were found not significantly different under the same stimulus. The cutoff 4.5 achieved specificity 71% and sensitivity 64% (p=0.04523). These findings indicate that ASC is also more sensitive and specific of an ET feature than TFT and pupil size to differentiate ASD from non-ASD. With reference to the scenario of the silently talking woman's face, NT people may quickly the mouth area and eyes back and forth (switches) to figure out what she is talking about instead of constantly focusing on eyes or mouth area; this could be referred to as “mind reading.” ASD individuals are less capable of or less interested in mind reading or theory of mind (ToM); therefore, the switches from eyes to mouth are significantly less than those of non-ASD. TOM is the human ability to perceive, interpret, and attribute the mental states of other people, and the alteration of this cognitive function is a core symptom of ASD.

The other finding is the consistently reduced pupil size in ASD across all the AOIs of different videos in at least a subset of the scenarios. Comparative examples reported significantly smaller baseline pupil size in the ASD group relative to matched controls. Pupil dilation is determined by emotional arousal. Pupil dilation metrics correlate with individual differences measured by the Social Responsiveness Scale (SRS), a quantitative measure of autism traits. Other comparative examples indicated that ASD individuals' pupils initially contracted more, took longer to dilate, and eventually dilated more than those of non-autistic participants. The amount that autistic people's pupils widen (dilate) when watching others interact and the speed at which this happens is linked with their understanding of social behavior.

The above-described study also demonstrated consistently reduced fixation time in ASD group vs non-ASD group Fixation durations were shortest in those infants who went on to receive an ASD diagnosis at 36 months, which makes fixation time a potential early marker for ASD. Saccades were found to be quite variable in this study. Saccades are rapid and simultaneous eye movements between two or more phases of fixation in the same direction designed to shift the fovea to objects of visual interest. Within ASD, saccade features correlated with measures of restricted and repetitive behavior. Decreased saccade amplitude and duration indicate spatially clustered fixations that attenuate visual exploration and emphasize endogenous over exogenous attention. ASD children made more saccades, slowing their reaction times; however, exogenous, and endogenous orienting, including gaze cueing, appear intact in ASD. Saccades of individuals with ASD were characterized by reduced accuracy, elevated variability in accuracy across trials, and reduced peak velocity and prolonged duration. At birth, infants can direct their gaze to interesting sights in the environment, primarily using saccadic eye movements. These rapid fixation shifts from one location to another are variable in newborns and often involve several hypo metric saccades that successively bring an object of interest closer to the infant's focal point. The results revealed that the number of fixations, fixation duration, number of saccades, saccade duration, saccade accuracy, and saccade latency did not differ significantly across groups. differences in gaze behavior of children with ASD, which are likely due to atypical social preferences rather than impaired control of eye movements and not due to underlying oculomotor deficiencies.

Conclusions: The study developed a set of eye tracking metrics included favored AOI shifts, AOI vacancy counts, AOI switches and found them significantly more sensitive and specific than metrics used in comparative examples, such as fixation time, to differentiate ASD from those non-ASD. This study further explored the correlation of these new metrics with ADOS-2 subscales to indicate the screening and diagnostic values for ASD using eye tracking.

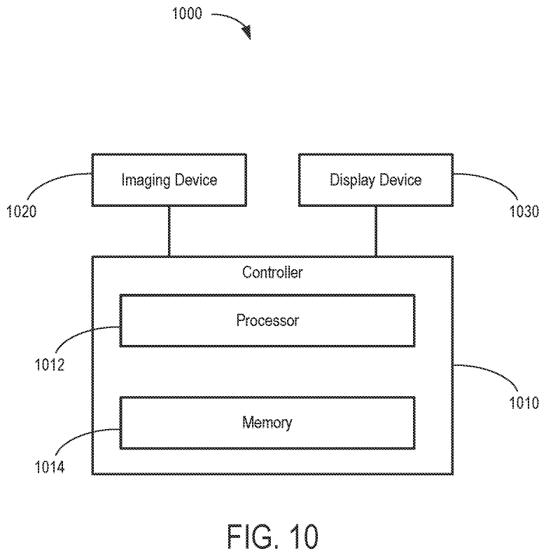

Referring to FIG. 10, an eye tracking system 100 (an example of an “eye tracking system” in accordance with the present disclosure) for screening for ASD.

For example, as illustrated in FIG. 10 an eye tracking system 1000 may include a controller 1010 having one or more inputs, processors, memories, and outputs.

The eye tracking system 1000 may include, access, or communicate with one or more user interfaces and/or an imaging device 1020, by way of a wired or wireless connection to the inputs. In various implementations, the eye tracking system 1000 may include any computing device, apparatus or system configured for carrying out instructions and providing input/output capabilities, and may operate as part of, or in collaboration with other computing devices and sensors/detectors (local and remote). In this regard, the eye tracking system 1000 may be a system that is designed to integrate a variety of software and hardware capabilities and functionalities, and/or may be capable of operating autonomously.

The input may include any one or more different input elements, such as a mouse, keyboard, touchpad, touch screen, buttons, and the like, for receiving various selections and operational instructions from a user through touch, movement, speech, etc. The input may also include various drives and receptacles, such as flash-drives, USB drives, CD/DVD drives, and other computer-readable medium receptacles, for receiving various data and information. To this end, input may also include various communication ports and modules, such as Ethernet, Bluetooth, or Wi-Fi, for exchanging data and information with these, and other external computers, systems, devices, machines, mainframes, servers or networks.

In addition to being configured to carry out various steps for operating the hair transplant system, the processor 1012 may be configured to execute instructions, stored in the memory 1014 in a non-transitory computer-readable media. The instructions executable by the processor 1012 may correspond to various instruction for completing a hair transplant procedure (such as those previously described). The memory 1014 may be or include a non-volatile medium, e.g., a magnetic media or hard disk, optical storage, or flash memory; a volatile medium, such as system memory, e.g., random access memory (RAM) such as dynamic RAM (DRAM), synchronous dynamic RAM (SDRAM), static RAM (SRAM), extended data out (EDO) DRAM, extreme data rate dynamic (XDR) RAM, double data rate (DDR) SDRAM, etc.; on-chip memory; and/or an installation medium where appropriate, such as software media, e.g., a CD-ROM, or floppy disks, on which programs may be stored and/or data communications may be buffered. Although the non-transitory computer-readable media can be included in the memory 1014, it may be appreciated that instructions executable by the processor 1012 may be additionally or alternatively stored in another data storage location having non-transitory computer-readable media. For example, the hair transplant system 1200 may be configured to implement cloud storage.

As used herein, a “processor” may include one or more individual electronic processors, each of which may include one or more processing cores, and/or one or more programmable hardware elements. The processor may be or include any type of electronic processing device, including but not limited to central processing units (CPUs), graphics processing units (GPUs), application-specific integrated circuits (ASICs), field-programmable gate arrays (FPGAs), microcontrollers, digital signal processors (DSPs), or other devices capable of executing software instructions. When a device is referred to as “including a processor,” one or all of the individual electronic processors may be external to the device (e.g., to implement cloud or distributed computing). In implementations where a device has multiple processors and/or multiple processing cores, individual operations described herein may be performed by any one or more of the microprocessors or processing cores, in series or parallel, in any combination.

In some aspects, the processor 1012 may be configured to receive and process image data from a subject, such as a donor or a recipient, captured by the imaging system 1020 to identify hair follicles and hair follicle orientations within a donor site of the donor and/or to determine implantation locations and necessary implantation angles within a recipient site of the recipient. In some aspects, the processor 1012 may access information and data, including video signals, stored in or emitted by the imaging system 1020. In some aspects, the imaging system 1020 may acquire either a single image or a continuous video signal using, for example, a camera, an infrared scanning system, or any other image capturing or video recording device that can be used to periodically image and/or scan and/or continuously record the subject.

In some non-limiting examples, the imaging system 1020 can include a camera such as a standard complementary metal-oxide-semiconductor (CMOS) camera, and a charge-coupled device (CCD) camera, and the like. The display device 1030 can include a display configured to display video and/or still images, such as a liquid crystal display (LCD), an organic light-emitting display (OLED), and the like.

In some implementations, the controller 1010, the imaging device 1020, and the display device 1030 may be integrated into a single device. For example, the eye tracking system 1000 may be a laptop computer, a tablet computer, a notebook computer, a smartphone, a desktop computer, a personal digital assistant (PDA), and the like. In other implementations, the imaging device 1020 and/or the display device 1030 may be a separate device configured to connect to the controller 1010. For example, the imaging device 1020 may be a webcam connected to the controller 1010, and/or the display device 1030 may be an external display (e.g., an external monitor) connected to the controller 1010. In such examples, the connection may be either wired (e.g., via a Universal Serial Bus (USB) interface, a FireWire interface, a High-Definition Multimedia Interface (HDMI), a DisplayPort interface, and the like) or wireless (e.g., via a Wi-Fi interface, a Bluetooth interface, a Near Field Communication (NFC) interface, and the like).

The eye tracking system 1000 may be configured to implement the systems and methods described herein via a program that is installed on a device locally (e.g., an app) or via a program that is remotely located (e.g., via a web interface). In either case, the eye tracking system 1000 may be configured to present a graphical user interface (GUI) on the display device 1030 to display still and/or video images, to receive user inputs or selections, to present instructions to the user, and so on.

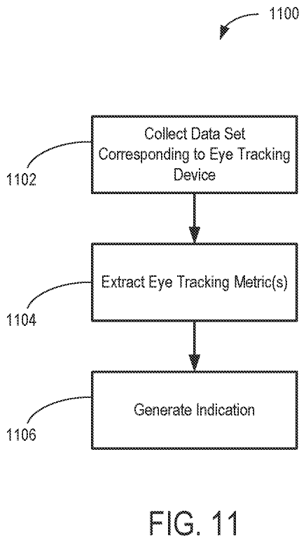

FIG. 11 illustrates an example method 1100 in accordance with the present disclosure. For purposes of illustration, the method 1100 is described as being performed by the system 1000. However, the present disclosure is not so limited and in some implementations, the method 1100 may be performed by another system (e.g., a server or other device that receives data from another system, such as the system 1000). The method 1100 may be performed for a subject, such as a human child.

The method 1100 includes an operation 1102 of collecting a data set corresponding to an eye tracking device. For example, the data set may be generated by the eye tracking device, and may be indicative of an individual's visual fixation with respect to a visual stimulus. The visual stimulus may include any one or more of the scenarios described above, such as the videos illustrated in FIGS. 1A, 4A, 7, and/or 8.

The method 1100 further includes an operation 1104 of extracting one or more eye metrics from the data set. The eye tracking metrics may include any combination of AOI switch incidences, AOI shift pathways, AOI vacancy incidences, total gaze points, and/or fixation counts. The metrics may be related to ASD core defects, including but not limited to repetitive and/or restrictive behaviors or social deficit.

The method 1100 further includes an operation 1106 of generating an indication based on the one or more eye tracking metrics. The indication may be at least one of a diagnosis or subtype of ASD. In some implementations, operations 1102-1106 may be performed by the processor of a system performing the method 1100 (e.g., on the processor 1012 of the controller 1010 of FIG. 10). In other implementations, operations 1102-1106 may be performed by another device based on data obtained by an eye tracking device. In still other implementations, some of operations 1102-1106 may be performed by the eye tracking device (e.g., collecting data using a camera) while others of operations 1102-1106 may be performed by the other device.

The operations of FIG. 11 need not be performed one after another in strict sequence. For example, operation 1102 may be performed continually or continuously to obtain an updating data set, and operation 1104 may be performed thereafter to extract the eye tracking metric or metrics.

Other examples and uses of the disclosed technology will be apparent to those having ordinary skill in the art upon consideration of the specification and practice of the invention disclosed herein. The specification and examples given should be considered exemplary only, and it is contemplated that the appended claims will cover any other such embodiments or modifications as fall within the true scope of the invention.

The Abstract accompanying this specification is provided to enable the United States Patent and Trademark Office and the public generally to determine quickly from a cursory inspection the nature and gist of the technical disclosure and in no way intended for defining, determining, or limiting the present invention or any of its embodiments.

What is claimed is:

1. A method for identifying a change in visual fixation of an individual over time, comprising:
collecting a data set collected indicative of an individual's visual fixation with respect to a visual stimulus as determined by an eye tracking device;
extracting one or more eye tracking metrics from the data set; and
generating, via software executing on a processor, an indication of at least one of a diagnosis or subtype indication based on the one or more eye tracking metrics.

2. The method of claim 1, wherein the one or more eye tracking metrics are at least one of an area of interest (AOI) switch incidence, AOI shift pathway, AOI vacancy incidence, a total gaze point, or a fixation count.

3. The method of claim 2, wherein the AOI switch incidence is related to autism spectrum disorder (ASD) core defects.

4. The method of claim 3, wherein the ASD core defects include repetitive and/or restrictive behaviors.

5. The method of claim 2, wherein the AOI vacancy incidence is a measure of shifts between favored and unfavored pathways and related to ASD core defects, including social deficit.

6. The method of claim 2, wherein the AOI shift pathway is related to ASD core defects.

7. The method of claim 1, further comprising generating a report including the diagnosis or subtype indication.

8. The method of claim 1, wherein the individual is a human child.

9. The method of claim 1, wherein the eye tracking device is a camera.

10. A system for identifying a change in visual fixation of an individual over time, comprising:
an eye tracking device configured to track eye movement of the subject while the subject watches a visual stimulus; and
a processor coupled with the eye tracking device and containing program instructions that, when executed, cause the system to:
collect a data set from the eye tracking device, the data set being indicative of an individual's visual fixation with respect to the visual stimulus,
extract one or more eye tracking metrics from the data set, and
generate an indication of at least one of a diagnosis or subtype of ASD based on the one or more eye tracking metrics.

11. The system of claim 10, wherein the one or more eye tracking metrics are selected from an area of interest (AOI) switch incidence, AOI shift pathway, AOI vacancy incidence, a total gaze point and a fixation count.

12. The system of claim 11, wherein the AOI switch incidence is related to ASD core defects.

13. The system of claim 12, wherein the ASD core defects include repetitive and/or restrictive behaviors.

14. The system of claim 11, wherein the AOI vacancy incidence is a measure of shifts between favored and unfavored pathways and related to ASD core defects.

15. The system of claim 14, wherein the ASD core defects include social deficit.

16. The system of claim 11, wherein the AOI shift pathway is related to ASD core defects.

17. The system of claim 10, further comprising a display device coupled with the processor, the display device being configured to display the visual stimulus.

18. The system of claim 10, wherein the eye tracking device is a camera.

19. A non-transitory computer-readable medium storing instructions that, when executed by a processor of a system, cause the system to perform operations comprising:
collecting a data set collected indicative of an individual's visual fixation with respect to a visual stimulus as determined by an eye tracking device;
extracting one or more eye tracking metrics from the data set; and
generating, via software executing on a processor, an indication of at least one of a diagnosis or subtype of ASD based on the one or more eye tracking metrics.

20. The non-transitory computer-readable medium of claim 19, wherein the one or more eye tracking metrics are at least one of an area of interest (AOI) switch incidence, AOI shift pathway, AOI vacancy incidence, a total gaze point, or a fixation count.
